(19) United States
(12) Patent Application Publication
(10) Pub. No.: US 2023/0355721 A1
(43) Pub. Date: Nov. 9, 2023

(54) DIETARY SUPPLEMENT COMPOSITION COMPRISING COLLAGEN

(71) Applicant: SOCIETE DES PRODUITS NESTLE S.A., Vevey (CH)

(72) Inventors: KURT SEIDENSTICKER, Chicago, IL (US); COREY FRIESE, Chicago, IL (US); KATIE COTE, Chicago, IL (US); CRISTINA CUTRI, Chicago, IL (US)

(21) Appl. No.: 17/759,572

(22) PCT Filed: Feb. 2, 2021
(86) PCT No.: PCT/EP2021/052381

§ 371 Date: Jul. 27, 2022

Related U.S. Application Data
(60) Provisional application No. 62/969,960, filed on Feb. 4, 2020.

(51) Int. Cl. A23L 33/16; A61K 31/198; A23L 2/39; A61K 31/145; A61K 31/4415; A23L 33/18; A61K 31/205; A23L 33/175; A61K 31/197; A61K 31/519; A61K 33/30; A61K 36/10; A61K 33/06; A61K 31/355; A61K 36/73; A61K 31/455; A61K 31/375; A23L 2/66; A61K 31/522; A61K 31/593; A61K 31/525; A61K 9/00; A61K 38/39; A23L 33/15; A61K 31/51; A61K 33/00

(52) U.S. Cl. CPC A61K 38/39; A23L 2/39; A23L 2/66; A23L 33/15; A23L 33/16; A23L 33/175; A23L 33/18; A61K 9/0053; A61K 31/145; A61K 31/197; A61K 31/198; A61K 31/205; A61K 31/355; A61K 31/375; A61K 31/4415; A61K 31/455; A61K 31/51; A61K 31/519; A61K 31/522; A61K 31/525; A61K 31/593; A61K 33/00; A61K 33/06; A61K 33/30; A61K 36/10; A61K 36/73

(57) ABSTRACT

The present invention is directed to improving physical and athletic performance and supporting recovery of joints, ligaments, and tendons with collagen peptides and dietary ingredients that physical and athletic performance. The dietary supplement composition of the present invention comprise collagen, preferably in the form of hydrolyzed collagen peptides, in combination with Branched-Chain Amino Acids (BCAA), Creatine Nitrate, Arginine Nitrate, N- acetyl-L-Carnitine, L-Citrulline, Betaine Anhydrous, Alpha-Glyceryl Phosphoryl Choline, Ancient Peat Extract with Apple Fruit Extract, and Caffeine. Physical and athletic performance, as well as recovery from exercise, is improved using this combination of components.

### FIELD OF THE INVENTION

The present invention is directed to improving physical and athletic performance and supporting recovery of joints, ligaments, and tendons with collagen peptides and dietary ingredients that physical and athletic performance. The dietary supplement composition of the present invention comprises collagen, preferably in the form of hydrolyzed collagen peptides, in combination with Branched-Chain Amino Acids (BCAA), Creatine Nitrate, Arginine Nitrate, N-acetyl-L-Carnitine, L-Citrulline, Betaine Anhydrous, Alpha-Glyceryl Phosphoryl Choline, Ancient Peat Extract with Apple Fruit Extract, and Caffeine. Physical and athletic performance, as well as recovery from exercise, is improved using this combination of components.

### BACKGROUND OF THE INVENTION

Modern athletes, and even average fitness enthusiasts are always looking for an edge in the gym or on the field, whether it is shaving off a few seconds on a mile run or getting an extra set in at the gym. The ingestion of nutritional beverages or drink powder mixes immediately preceding physical activity has been a preferred method to help enhance performance and put the body in an optimal state.

In addition to a need for optimizing performance, consuming collagen peptides as a dietary supplement has been associated with enhanced recovery from exercise and provides amino acids to support muscles, joints, ligaments, and tendons that can be stressed or broken down during exercise or physical activity (Chang et al. 2014, Clifford et al. 2019).

While adequate nutrition, sleep, and a healthy lifestyle are the core of optimizing performance, certain ingestible dietary supplements and ingredients have the potential to maximize power output and increase energy, endurance, and focus. These desirable parameters can be enhanced by improving circulation, increasing ATP energy, maintaining cellular hydration, and providing nutrients that improve cognitive function. Research has shown that several dietary supplement ingredients have inherit properties that promote these performance enhancing functions such as Creatine Nitrate, Arginine Nitrate, L-Citrulline, Betaine Anhydrous, Alpha-Glyceryl Phosphoryl Choline, Ancient Peat Extract with Apple Fruit Extract, and Caffeine.

However, there remains a need to improve performance and increase the uptake of collagen. The present invention resolves this deficiency and enhances the uptake of collagen peptides and improves athletic performance based on the components of the composition.

### SUMMARY OF THE INVENTION

The present invention is directed to a dietary supplement composition comprising collagen peptides, Branched-Chain Amino Acids (BCAA), Creatine Nitrate, Arginine Nitrate, N-acetyl-L-Carnitine, L-Citrulline, Betaine Anhydrous, Alpha-Glyceryl Phosphoryl Choline, Ancient Peat Extract with Apple Fruit Extract, and Caffeine. The composition may further include vitamins including Vitamin C, Niacin, Vitamin B6, or Pantothenic Acid.

In one embodiment, the collagen is present as collagen peptides in an amount of about 15 to about 22 weight percent of the composition, Branched Chain Amino Acids (BCAA) which include Leucine, Isoleucine, Valine are present in an amount of about 15 to about 22 weight percent of the composition, L-Citrulline is present in an amount of about 9 to about 13 weight percent of the composition, Betaine Anhydrous is present in an amount of about 7 to about 11 weight percent of the composition, Creatine Nitrate is present in an amount of about 4.6 to about 6.5 weight percent of the composition, Arginine Nitrate is present in an amount of about 4.6 to about 6.5 weight percent of the composition, N-Acetyl Carnitine is present in an amount of about 1.5 to about 2.2 weight percent of the composition, A blend of Ancient Peat Extract and Apple Fruit Extract is present in an amount of 0.45 to about 0.65 weight percent, and Caffeine is present in an amount of about 0.45 to about 0.65 weight percent of the composition.

The composition of the present invention may be in the form of a powder or liquid.

The compositions of the present invention are intended for use to replace collagen broken down during physical activity and to improve physical and athletic performance.

### DETAILED DESCRIPTION OF THE INVENTION

The present invention relates to a dietary supplement composition that may be consumed as a supplement to maintain collagen levels in the body, which help to support muscles, joints, tendons, and ligaments and to support healthy sleep and improve physical and athletic performance.

The compositions of the present invention comprise collagen, preferably in the form of hydrolyzed collagen peptides, in combination with Branched-Chain Amino Acids (BCAA), Creatine Nitrate, Arginine Nitrate, N-acetyl-L-Carnitine, L-Citrulline, Betaine Anhydrous, Alpha-Glyceryl Phosphoryl Choline, Ancient Peat Extract with Apple Fruit Extract, and Caffeine. The composition may be used in the form of a liquid, solution, suspension, powder, or gel. If in powder form, the powder is mixed in a soluble liquid prior to administration as directed.

Collagen is present in the composition in an amount of about 7 to 30 weight percent of the composition, about 11 to 26 weight percent of the composition, or about 15 to 22 weight percent of the composition. Alternatively, collagen may be present in the composition in an amount of about 2-8 grams/serving, about 3-7 grams/serving, or about 5 grams/serving. In a preferred embodiment, collagen may be present in the composition in an amount of about 5 grams/serving. Collagen is a protein and may be present in any available form in the composition. However, the present invention preferably uses collagen peptides. Collagen peptides may be sourced from any known source, including but not limited to, beef, chicken, fish, eggshell membrane, and chicken egg whites. In one preferred embodiment, collagen peptides are in the form of marine collagen, preferably from white fish.

Essential Amino Acids which are amino acids that humans cannot synthesize on their own. These are phenylalanine, valine, threonine, tryptophan, methionine, leucine, isoleucine, lysine, and histidine. These may be present in the composition in an amount of about 7-30 weight percent, about 11-26 weight percent, or about 15-22 weight percent.

Branched Chain Amino Acids (BCAA) which are leucine, isoleucine and valine are present in the composition in an amount of about 2-8 g/serving, about 3-7 g/serving, about 4-6 g/serving, or about 5 g/serving. Alternatively, Branched Chain Amino Acids (BCAA) may be present in the composition in an amount of about 7-30 weight percent, about 11-26 weight percent, or about 15-22 weight percent.

L-Citrulline is also present in the composition in an amount of about 1-5 g/serving, about 2-4 g/serving, about 2.5-3.5 g/serving, or about 3 g/serving. Alternatively, L-Citrulline may be present in the composition in an amount of about 3.7-19 weight percent, about 7-15 weight percent, or about 9-13 weight percent.

Betaine Anhydrous is also present in the composition in an amount of about 1-5 g/serving, about 2-4 g/serving, about 2.5-3.5 g/serving, or about 3 g/serving. Alternatively, Betaine

Anhydrous may be present in the composition in an amount of about 3.7-19 weight percent, about 7-15 weight percent, or about 9-13 weight percent.

Creatine Nitrate is also present in the composition in an amount of about 0.5-3 g/serving, about 1-2 g/serving, about 1.25-1.75 g/serving, or about 1.5 g/serving. Alternatively, Creatine Nitrate may be present in the composition in an amount of about 1.8-11.1 weight percent, about 3.7-7.4 weight percent, or about 4.6-6.5 weight percent.

Arginine Nitrate is also present in the composition in an amount of about 0.5-3 g/serving, about 1-2 g/serving, about 1.25-1.75 g/serving, or about 1.5 g/serving. Alternatively, Arginine Nitrate may be present in the composition in an amount of about 1.8-11.1 weight percent, about 3.7-7.4 weight percent, or about 4.6-6.5 weight percent.

N-acetyl-L-Carnitine is also present in the composition in an amount of about 0.1-1.5 g/serving, about 0.25-0.75 g/serving, about 0.4-0.6 g/serving, or about 0.5 g/serving. Alternatively, N-acetyl-L-Carnitine may be present in the composition in an amount of about 0.35-5.6 weight percent, about 0.9-2.8 weight percent, or about 1.45-2.25 weight percent.

A blend of ancient peat extract and apple fruit extract is also present in the composition in an amount of about 0.05-0.3 g/serving, about 0.1-0.2 g/serving, about 0.125-0.175 g/serving, or about 0.15 g/serving. Alternatively, a blend of ancient peat extract and apple fruit extract may be present in the composition in an amount of about 0.19-1.11 weight percent, about 0.37-0.74 weight percent, or about 0.46-0.65 weight percent.

Caffeine Is also present in the composition in an amount of about 0.05-0.3 g/serving, about 0.1-0.2 g/serving, about 0.125-0.175 g/serving, or about 0.14 g/serving. Alternatively, Caffeine may be present in the composition in an amount of about 0.19-1.11 weight percent, about 0.37-0.74 weight percent, or about 0.46-0.65 weight percent.

Vitamins may further be added to the composition. Any vitamins suitable for ingestion in a dietary supplement is suitable for use. Suitable vitamins include but are not limited to Vitamin B, including Vitamin B1 (e.g., as thiamine chloride), Vitamin B2 (e.g., as riboflavin), Vitamin B3 (e.g., niacinamide), Vitamin B6 (e.g., as pyridoxine HCl), Vitamin B5 (e.g., as pantothenic acid or D-calcium pantothenate), Vitamin B12 (cyanocobalamin), folate (e.g., as folic acid), Vitamin C (e.g., as sodium ascorbate), Vitamin D, Vitamin E, etc. In the context of the present invention, each of the vitamins may be independently present in the composition in an amount of about 0-0.2 grams/serving, about 0-1 grams/serving, about 0.2-0.8 grams/serving, 0.4-0.6 grams/serving, or about 0-0.25, about 0-0.2, about 0.001-0.2, or about 0.002-0.175 weight percent of the total composition.

Moreover, the composition may be flavored using any known natural flavors to enhance the flavor of the composition. Additionally, other additives suitable for dietary supplements, including but not limited to, monk fruit extract, Himalayan pink sea salt, fruit and vegetable juice powder, citric acid, and malic acid

In accordance with the present invention, suitable compositions include, but are not limited, to those described in Table 1.

TABLE 1

| | | |
|---|---|---|
| Ingredient | Amount per serving | % Daily Value |
| Protein | 5 g | ** |
| Vitamin C | 45 mg | 50% |
| Niacin (niacinamide) | 8 mg NE | 50% |
| Vitamin B6 (Pyridoxine HCl) | 1.7 mg | 100% |
| Pantothenic Acid | 2.5 mg | 50% |
| Sodium | 120 mg | 5% |
| Collagen Peptides (from bovine) | 5 g | ** |
| 3:1:1 BCAA Blend (L-Leucine, L-Isoleucine, L-Valine) | 5 g | ** |
| Betaine anhydrous | 2.5 g | ** |
| Creatine Nitrate | 1.5 g | ** |
| Arginine Nitrate | 1.5 g | ** |
| N-acetyl-L-Carnitine | 500 mg | ** |
| Alpha-Glyceryl Phosphoryl Choline | 150 mg | ** |
| Ancient Peat Extract and Apple Fruit Extract | 150 mg | ** |
| Caffeine | 140 mg | ** |

**Daily Value Not Established

In an embodiment of the present invention, the caloric intake of the supplement is less than about 100 calories, less than about 75 calories, or about 40 calories per serving. Additionally, the amount of carbohydrates and total sugars in the composition is desirably low. For example, the carbohydrates may be present in an amount of less than 10 grams/serving, less than 7 grams/serving, less than 5 grams/serving, about 4 grams/serving, or about 2 grams/serving. Similarly, the total sugars is desirably kept low, e.g., less than 6 grams/serving, less than 5 grams/serving, or about 0 gram/serving. As noted above, the content of the protein, in the form of collagen or collagen peptides, is high, preferably above 2 grams/serving, above 4 gram/serving, or about 5 grams/serving.

### Example 1

A composition of the formula depicted in Table 1 was prepared.

### REFERENCES

Chang et al., (2014) Molecular biomechanics of collagen molecules, Materials Today,Volume 17, Number 2, March 2014.

Clifford et al., (2019) The effects of collagen peptides on muscle damage, inflammation and bone turnover following exercise: a randomized, controlled trial, Amino Acids (2019) 51:691-704.

1. A dietary supplement composition comprising collagen peptides in an amount of about 30 to 60 weight percent of the composition, glutamine in an amount of about 2 to about 3.5 weight percent of the composition, and taurine in an amount of about 0.2 to about 0.5 weight percent of the composition.

2. The dietary supplement composition of claim 1, wherein the composition further comprises at least one nutritionally essential amino acid selected from the group consisting of: leucine, isoleucine, valine, lysine, phenylalanine, threonine, tryptophan, histidine, and methionine in an amount of about 15-22 weight percent of the composition.

3. The dietary supplement according to claim 2 wherein the composition comprises at least one branched chain amino acid (BCCA) selected from the group consisting of: leucine, isoleucine, and valine in an amount of about 15-22 weight percent of the composition.

4. The dietary supplement according to claim 2 wherein the total amount of essential amino acids present is in an amount of about 7-30 weight percent.

5. The dietary supplement composition of claim 2, wherein the composition is a powder.

6. The dietary supplement composition of claim 2, wherein the composition is a liquid.

7. The dietary supplement composition of claim 2, wherein collagen is present in an amount of about 40-55 weight percent of the composition, glutamine is present in an amount of 2.75 to about 3.0 weight percent of the composition, and taurine is present in an amount of about 0.3-0.45 weight percent of the composition.

8. The dietary supplement composition of claim 2, wherein collagen is present in an amount of 50-55 weight percent of the composition, glutamine is present in an amount of about 2.75-3.0 weight percent of the composition, and taurine is present in an amount of about 0.4-0.42 weight percent of the composition.

9. The dietary supplement composition of claim 2, wherein the composition further comprises coconut water in an amount of about 10 to 20 weight percent of the composition.

10. The dietary supplement composition of claim 2, wherein the composition further comprises coconut water in an amount of about 12 to 16 weight percent of the composition.

11. The dietary supplement composition of claim 2, wherein the composition further comprises electrolytes selected from the group consisting of sodium, potassium, magnesium, and zinc.

12. The dietary supplement composition of claim 2, further comprising electrolytes selected from the group consisting of: sodium, potassium, magnesium, calcium, bicarbonate, chloride, and phosphate.

13. The dietary supplement composition of claim 2, further comprising selenium.

14. The dietary supplement composition of claim 2, further comprising one or more of the following vitamins: Vitamin B1, Vitamin B2, Vitamin B3, Vitamin B6, Vitamin B5, folate, Vitamin C, Vitamin D, and Vitamin E.

15. The dietary supplement composition of claim 14, wherein the composition comprises each of the following vitamins: Vitamin B1, Vitamin B2, Vitamin B3, Vitamin B6, Vitamin B5, folate, Vitamin C, and Vitamin D.

16. A dietary supplement composition comprising the following ingredients in the amounts:

| | | |
|---|---|---|
| Ingredient | Amount per serving | % Daily Value |
| Protein | 5 g | Unknown |
| Vitamin C | 45 mg | 50% |
| Niacin (niacinamide) | 8 mg NE | 50% |
| Vitamin B6 (Pyridoxine HCl) | 1.7 mg | 100% |
| Pantothenic Acid | 2.5 mg | 50% |
| Sodium | 120 mg | 5% |
| Collagen Peptides (from bovine) | 5 g | Unknown |
| 3:1:1 BCAA Blend (L-Leucine, L-Isoleucine, L-Valine) | 5 g | Unknown |
| Betaine anhydrous | 2.5 g | Unknown |
| Creatine Nitrate | 1.5 g | Unknown |
| Arginine Nitrate | 1.5 g | Unknown |
| N-acetyl-L-Carnitine | 500 mg | Unknown |
| Alpha-Glyceryl Phosphoryl Choline | 150 mg | Unknown |
| Ancient Peat Extract and Apple Fruit Extract | 150 mg | Unknown |
| Caffeine | 140 mg | Unknown |

.

17. A method of replacing collagen broken down during physical activity comprising administering to a patient in need thereof a composition comprising a dietary supplement composition comprising collagen peptides in an amount of about 30 to 60 weight percent of the composition, glutamine in an amount of about 2 to about 3.5 weight percent of the composition, and taurine in an amount of about 0.2 to about 0.5 weight percent of the composition following said physical activity.
